(19) United States
(12) Patent Application Publication
(10) Pub. No.: US 2014/0086888 A1
(43) Pub. Date: Mar. 27, 2014

(54) EBV-SPECIFIC CYTOTOXIC T-LYMPHOCYTES FOR THE TREATMENT OF LOCOREGIONAL NASOPHARYNGEAL CARCINOMA (NPC)

(71) Applicants: Helen E. Heslop, Houston, TX (US); Karin Straathof, London (GB); Stephen M.G. Gottschalk, Houston, TX (US); Cliona M. Rooney, Bellaire, TX (US); Chrystal U. Louis, Houston, TX (US); Malcolm Brenner, Houston, TX (US)

(72) Inventors: Helen E. Heslop, Houston, TX (US); Karin Straathof, London (GB); Stephen M.G. Gottschalk, Houston, TX (US); Cliona M. Rooney, Bellaire, TX (US); Chrystal U. Louis, Houston, TX (US); Malcolm Brenner, Houston, TX (US)

(21) Appl. No.: 13/820,169

(22) PCT Filed: Sep. 1, 2011
(86) PCT No.: PCT/US11/50221

§ 371 Date: Aug. 13, 2013

Related U.S. Application Data
(60) Provisional application No. 61/379,354, filed on Sep. 1, 2010.

(51) Int. Cl. A61K 39/245; A61K 45/06

(52) U.S. Cl. CPC A61K 39/245; A61K 45/06
USPC 424/93.71

(57) ABSTRACT

The present invention concerns methods and compositions for treatment of locoregional nasopharyngeal carcinoma utilizing EBV-specific T cells. In specific embodiments, locoregional solid tumors are treated with cytotoxic T lymphocytes specific for at least one EBV antigen, such as LMP2, LMP1, and/or EBNA1, for example.

This application claims priority to U.S. Provisional Patent Application Ser. No. 61/379,354, filed Sep. 1, 2010, which is incorporated by reference herein in its entirety.

### STATEMENT REGARDING FEDERALLY SPONSORED RESEARCH OR DEVELOPMENT

This invention was made with government support under P01 CA94237 awarded by NIH, by T32 DK64717 awarded by NIDDK, and by K12 CA09043306 awarded by NCI. The government has certain rights in the invention.

### TECHNICAL FIELD

The present invention generally concerns the fields of cell biology, molecular biology, and medicine. In particular, the field of the invention concerns immunotherapy for cancer.

### BACKGROUND OF THE INVENTION

Nasopharyngeal carcinoma (NPC) arises from the epithelial cells of the nasopharynx and almost all nonkeratinizing and undifferentiated forms of the tumor are associated with Epstein Barr Virus (EBV) (Chan et al., 2002; Raab-Traub, 2002) NPC patients with limited local disease have a good prognosis when treated with chemotherapy and intensity-modulated radiation therapy, but the patients with bulky locoregional or metastatic disease often relapse or have refractory disease. (Chan et al., 2002; Mould and Tai, 2002; Ayan et al., 2003) Historical 5-year overall survival rates for those with advanced stage III or IV disease are reported between 54% to 76% and 29% to 56%, respectively. (Chan et al., 2002; Mould and Tai, 2002; Lee et al., 2010; Cooper, 1997) The patients who survive experience severe short-term and longterm morbidity, with an associated profound impact on their quality-of-life. (Wang et al., 2000; Louis et al., 2007) Hence, there is a need for novel therapies that both improve disease-free survival and reduce treatment-related complications.

One promising approach is therapy with autologous EBV-specific cytotoxic T lymphocytes (EBV-CTLs) targeted to the EBV antigens EBNA1, latent membrane protein LMP1 and LMP2 expressed by most NPC tumors. (Chan et al., 2002; Raab-Traub, 2002) Although adoptive immunotherapy with EBVCTLs for NPC has shown evidence of clinical activity in Phase I dose escalation studies, (Straathof et al., 2005; Comoli et al., 2005) at present the overall response rate and most appropriate patient population (i.e., those with locoregional as compared with metastatic, relapsed/refractory disease) is unclear. It has also been unclear whether antitumor efficacy is influenced by the particular EBV antigens the infused lines are primarily directed toward, or by the degree of in vivo expansion measured within the peripheral blood, as observed for other T-cell therapeutics. (Rosenberg et al., 2008; Rosenberg and Dudley, 2009) This information is useful for developing further CTL applications and improving clinical progress.

### BRIEF SUMMARY OF THE INVENTION

In general, the present invention concerns immunotherapy of cancer, including nasopharyngeal carcinoma, for example. In particular cases an individual has been diagnosed with NPC and is administered methods and/or compositions as described herein. In specific embodiments, there is a method of treating an individual with locoregional nasopharyngeal carcinoma by administering (such as by infusion) cytotoxic T lymphocytes (CTLs) that are specific to at least one Epstein-Barr virus (EBV) antigen. The individual may be given multiple administrations of the CTLs encompassed by the invention, for example with two or more separate infusions of EBV CTLs, including 3, 4, 5, 6, 7, 8, 9, 10, or more infusions.

A skilled artisan recognizes that the major antigens of EBV include six EBV-associated nuclear antigens (EBNA, 1,2,3a,3b,3c,-leader protein (LP)), early antigen (EA), which comes in diffuse (D) and restricted (R) forms, viral capsid antigen (VCA), EBV-induced membrane antigen (MA), and latent membrane proteins (LMP). In certain cases, the CTLs recognize one or more EBV antigens, including LMP 1; LMP2; EBNA; EBNA, His; EA, p18; p18,GST; p18M; p23; and p23 His, for example.

In some embodiments of the invention, EBV-specific CTLs are generated using the procedures that involve employing autologous LCLs as antigen-presenting cells. In certain embodiments, EBV-CTLs for NPC applications focus on subdominant antigens, such as LMP1, LMP2, and EBNA1, for example. An individual may enrich the EBV-CTL for particular antigens, such as LMP1 and LMP2, for example. An individual may enrich the EBV-CTL for particular subdominant antigens.

In certain embodiments of the invention, the methods include treatment of NPC that is initial, recurrent, or refractive. The individual being treated may be a child, adolescent, or adult and may be male or female.

In certain embodiments of the invention, the methods of the invention are employed in conjunction with other cancer treatments for NPC, including radiation therapy, surgery, and/or chemotherapy, for example. The combination treatments may be administered concomitantly or separately, and their effects may be additive or synergistic.

In some embodiments of the invention, single or multiple doses are delivered to the individual. In some embodiments, the dosages range from 1×10⁷ CTL/m² to 1×10⁹ CTL/m² or 1×10⁷ to 1×10⁸ CTL/m², for example.

The foregoing has outlined some of the features and technical advantages of the present invention in order that the detailed description of the invention that follows may be better understood. Additional features and advantages of the invention will be described hereinafter which form the subject of the claims of the invention.

### BRIEF DESCRIPTION OF THE DRAWINGS

FIG. 1 shows transient increase of LMP2-specific T-cell frequency after EBV-CTL infusion. The frequency of (A) EBV-specific, (B) LMP2-specific, and (C) CMV-specific T cells was determined using ELIspot assays. There was a trend toward increased LMP2-specific T cells (P=0.081) at 2 weeks after CTL infusion (- -: individual patients; —: median; NED: no evidence of disease).

FIG. 2 demonstrates clinical response after EBV-CTL infusion. PET images of patient (P1668) before and 8 weeks after EBV-specific CTL infusion are provided.

FIG. 3 shows overall and progression-free survival after EBV-CTL infusion. A, Overall survival, (B) Overall survival by disease status at first CTL infusion, (C) Progression-free survival, (D) Progression by disease status at first CTL infusion (P value: metastatic disease vs. remission).

### DETAILED DESCRIPTION OF THE INVENTION

In the following description, certain details are set forth such as specific quantities, sizes, etc. so as to provide a thorough understanding of the present embodiments disclosed herein. However, it will be obvious to those skilled in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

The present application incorporates both U.S. patent application Ser. No. 12/862,409, filed Aug. 24, 2010, and U.S. Provisional Patent Application Ser. No. 61/236,261, filed Aug. 24, 2009, by reference herein in their entirety.

### I. CERTAIN EMBODIMENTS OF THE INVENTION

Patients with recurrent or refractory Epstein Barr Virus (EBV)-positive nasopharyngeal carcinoma (NPC) continue to have poor outcomes. Earlier Phase I dose escalation clinical study of 10 NPC patients showed that infusion of EBV-specific cytotoxic T cells (EBV-CTLs) was safe and had antitumor activity. To better define the overall response rate and discover whether disease status, EBV-antigen specificity, and/or in vivo expansion of infused EBV-CTLs predicted outcome, 13 additional NPC patients were treated with EBV-CTLs in a fixed-dose, Phase II component of the study. Toxicity, efficacy, specificity, and expansion of infused CTLs was tested for all 23 recurrent/refractory NPC patients treated on this Phase I/II clinical study. At the time of CTL infusion, 8 relapsed NPC patients were in remission and 15 had active disease. No significant toxicity was observed. Of the relapsed patients treated in their second or subsequent remission, 62% (5/8) remain disease free (at 17 to 75 mo), whereas 48.7% (7/15) of those with active disease had a CR/CRu (33.3%) or PR (15.4%). In contrast to locoregional disease, metastatic disease was associated with an increased risk of disease progression (HR: 3.91, P=0.015) and decreased overall survival (HR: 5.55, P=0.022). Neither the specificity of the infused CTLs for particular EBV antigens nor their measurable in vivo expansion discernibly influenced outcome. Treatment of patients with relapsed/refractory EBV-positive NPC with EBV-CTLs is safe and is associated with significant, long-term clinical benefit for patients with locoregional disease.

### II. NASOPHARYNGEAL CARCINOMA

Nasopharyngeal carcinoma (NPC) is cancer that occurs in the nasopharynx, which is the narrow tubular passage behind the nasal cavity and above the back of the throat. It may be asymptomatic, especially in early stages, or it may have one or more of the following symptoms, in some cases: a lump in the neck caused by a swollen lymph node; blood in the saliva; bloody nasal discharge, nasal congestion, hearing loss, frequent ear infections, and headaches. Risk factors include sex (more common in women), race (Asian or north African descent), age (between ages of 30 and 50), salt-cured foods (chemicals released upon cooking), EBV, family history, smoking, and alchohol.

Nasopharyngeal carcinoma frequently spreads (metastasizes) beyond the nasopharynx. Most people with nasopharyngeal carcinoma have regional metastases, meaning cancer cells from the initial tumor have migrated to nearby areas, such as lymph nodes in the neck. Cancer cells that spread to other areas of the body (distant metastases) most commonly travel to the bones and bone marrow, lungs and liver.

Locoregional NPC is defined as carcinoma that is either confined to the nasopharynx or that has only regionally metastasized, such as to nearby areas including lymph nodes in the neck, for example (but not metastasis to the bone or liver).

Diagnostic procedures may include physical exam (such as feeling for swelling in lymph nodes), nasal endoscopy, and/or biopsy. Upon definitive identification of cancer, one can perform further tests, such as CT, MRI, PET, and/or X-ray, for example to determine the stage of the cancer. Treatment for NPC in the art presently includes radiation therapy, surgery, and/or chemotherapy. Chemotherapy may include, for example, cisplatin, gemcitabine, bleomycin, methotrexate, leucovorin, vincristine, and/or fluorouracil.

### III. EXAMPLES

The following examples are presented in order to more fully illustrate the preferred embodiments of the invention. They should in no way, however, be construed as limiting the broad scope of the invention.

### Example 1

### Exemplary Materials and Methods

### Participants

This study was approved by the Institutional Review Board at Baylor

College of Medicine and The Methodist Hospital, and by the Food and Drug Administration. The patients were eligible for the study if they had been treated for either advanced stage [stage III or IV disease according to the American Joint Committee on Cancer (AJCC) staging system] or had relapsed/refractory EBV-positive NPC. EBV-positivity was determined by in situ hybridization or polymerase chain reaction (PCR) amplification for EBV-encoded RNAs (EBER). Patients had to have received no therapy or experimental agents for at least 4 weeks before study entry, and recovered from treatment related acute adverse effects. The patients had to have a life expectancy more than 6 weeks, Karnofsy score Z50, and no evidence of pregnancy or intercurrent infection. Laboratory parameters included bilirubin and creatinine values <2 times normal, aspartate aminotransferase levels <3 times normal, and a hemoglobin level more than 8.0 g/dL. All patients were treated between July of 2002 and August of 2007.

### Generation of EBV-transformed Lymphoblastoid Cell Lines and EBV-CTLs

Autologous lymphoblastoid cell lines (LCL) and EBV-CTLs were generated according to current Good Manufacturing Practice (cGMP) guidelines as described earlier.^{9,13,14} After expansion, EBV-CTLs were tested for sterility, human leukocyte antigen (HLA)-identity, immunophenotype, and EBV-specificity at the time of cryopreservation. Specificity was tested in a 4-hour ⁵¹Chromium release cytotoxicity assay.

### Study Description

All patients had imaging with magnetic resonance imaging (MRI) and/or fluorodeoxyglucose positron emission tomography (PET) to assess overall disease burden before CTL infusion. The patients treated on the Phase I portion of the study were treated on one of 3 dose levels and received either 2 doses of 2×10⁷ CTL/m² (dose level 1), 1 dose of 2×10⁷ and 1 dose of 1×10⁸ CTL/m² (dose level 2), or 1 dose of 1×10⁸ and 1 dose of 2×10⁸ CTL/m² (dose level 3). All patients treated on the Phase II extension were treated at the highest dose level. CTL infusions were given 2 weeks apart. Peripheral blood samples were obtained before CTL infusion and at predetermined time points after infusion to evaluate for toxicity and EBV immunity. The clinical response to CTL was assessed by radiographic imaging 8 weeks after the last infusion date (or earlier if indicated). Patients with either a partial response (PR) or stable disease (SD) at the time of reimaging were eligible to receive additional CTL infusions. Longterm follow-up continued until July 2009. Although initial data from 10 patients treated on the Phase I portion of this clinical trial has been published earlier, 9 updated information related to clinical status and CTL product analysis is included within the results data detailed below. All imaging studies were rereviewed, 1 patient (815), who was earlier reported as having no disease, is now reported as having imaging findings of unknown significance.

### Clinical Response Criteria

Clinical response to CTL infusion was evaluated by comparing disease identified by MRI and/or FDG-PET radiographs obtained preinfusion to those obtained 8 weeks postinfusion, or as clinically indicated. FDG-PET imaging was used as additional conformation of response, as many patients have residual masses after standard therapy that may not represent active disease. Furthermore, as this study was open to children, rebiopsy of residual masses was not mandatory for study participation. The patients in remission at the time of infusion were defined as having no evidence of disease (NED) if subsequent scans had no new lesions. The patients with the abnormalities of unknown significance (i.e., infection vs. disease) whose subsequent scans had no radiographic evidence of abnormal lesions were considered a complete response undetermined (CRu). All other responses, including CR, PR, SD, and PD, were determined using Response Evaluation Criteria in Solid Tumors (RECIST). (Therasse et al., 2000)

### Enzyme-linked Immunospot (ELIspot) Assay

The frequency of EBV-specific, LMP1-specific, and LMP2-specific T cells in the CTL product and peripheral blood of patients was measured using interferon-γ (IFN-γ) ELIspot assays as described earlier. (Louis et al., 2009; Gottschalk et al., 2003) CTL or PBMCs were stimulated with HLA-restricted peptides derived from EBV antigens (Genemed Synthesis, San Antonio, Tex.) or overlapping peptide mixes for BZLF1, EBNA1, EBNA3A, EBNA3B, EBNA3C, LMP1, and LMP2 were used in ELIspot assays. (Straathof et al., 2005; Hislop et al., 2007; Khanna and Burows, 2000). Peptide mixes contained 15 amino-acid peptides covering the entire length of the corresponding protein with an 11 amino-acid overlap (JPT Peptide Technologies, Berlin, Germany). The frequency of EBVspecific, LMP1-specific, and LMP2-specific T cells in the peripheral blood of patients was determined by using autologous LCL or LMP1, and LMP2 peptide mixes. For cytomegalovirus (CMV)-seropositive patients, the frequency of CMV pp 65 protein (CMVpp65)-specific T cells was determined using a CMVpp65 peptide mix (JPT Peptide Technologies).

### Flow Cytometry

A FACScalibur instrument (Becton Dickinson, San Jose, Calif.) and CellQuest software (Becton Dickinson) was used for flow cytometric analysis. Monoclonal antibodies were obtained from Becton Dickinson and included anti-CD3, -CD4, -CD8, -CD16, -CD19, -CD27, -CD28, -CD45R0, CD45RA, -CD56, -TCRα/β, and -TCRγ/δ. Negative controls included isotype antibodies.

### Statistical Analysis

The Phase I/II study was designed to be a dose escalation investigation leading to a fixed-dose Phase II study. Four patients were treated on dose level 1 (1 compassionate care), 3 patients on dose level 2, and 16 patients on (fixed) dose level 3. No dose-limiting toxicity was observed, and all patients were included in the analysis. The descriptive statistics were used to evaluate standard demographic and clinical variables. Biological responses by EBV-specific, LMP2-specific, and CMV-specific T cells were analyzed for their changes from the baseline (CTL infusion) to 2 weeks and 6 weeks post infusion, respectively, using the nonparametric Wilcoxon signed-rank test. The survival curves were constructed using the Kaplan-Meier method and compared using the weighted log-rank test.

The patient data were censored at the time of death or if enrolled on another therapeutic/experimental trial, or if lost to follow-up. An univariate Cox regression was used to determine clinical factors significantly associated with survival. A P value less than 0.05 was considered statistically significant.

### Example 2

### Patient Characteristics

The clinical and disease-specific characteristics of the 23 patients are listed in Tables 1 and 2. A majority of patients were male, White, and had either AJCC Stage III or Stage 1V disease at the time of initial diagnosis.

TABLE 1
Characteristics of Enrolled Study Population

| | | |
|---|---|---|
| Sex (%) | | |
| Male | 17 | (73.9) |
| Female | 6 | (26.1) |
| Mean age at enrollment, years (range) | 29.2 | (11-63) |
| Ethnic origin (%) | | |
| African-American | 6 | (26.1) |
| Asian | 4 | (17.4) |
| White | 11 | (47.8) |
| Hispanic | 1 | (4.4) |
| Native American | 1 | (4.4) |
| Disease status at first CTL infusion (%) | | |
| Remission | 8 | (34.8) |
| Local-Regional | 3 | (13.0) |
| Unknown | 2 | (8.7) |
| Metastatic | 10 | (43.5) |
| Mean number of chemotherapy regimens before | 2.6 | (1-6) |
| stady enrollment (range) | | |

TABLE 2
Detailed Patient Characteristics and Outcome

| | | | | Prior | | Site of | CTL* | |
|---|---|---|---|---|---|---|---|---|
| | Age and | | Stage at | Therapy | | Disease at | (Dose | Response to Therapy |
| P# | Sex at Infusion | | Diagnosis | CT | XRT | Infusion | Level) | (Duration in mo) |
| 729 | 50 | F | IV: T4N3bM0 | 1 | 1 | NED | 2 (1) | Remains in remission (>80 mo); |
| | | | | | | | | alive |
| 606 | 59 | F | III: T3N2M0 | 1 | 1 | NED | 2 (1) | Recurrent disease (60 mo); alive |
| 697 | 11 | F | III: T1N2M0 | 1 | 1 | NED | 2 (1) | Remains in remission (>80 mo); |
| | | | | | | | | alive |
| 1642 | 11 | M | IV: TXNXM1 | 3 | 1 | NED | 2 (3) | Remains in remission (>42 mo); |
| | | | | | | | | alive |
| 1946 | 63 | F | III: T3NTM0 | 1 | 1 | NED | 2 (3) | Remains in remission (>28 mo); |
| | | | | | | | | alive |
| 1969 | 38 | M | III: T3N0M0 | 2 | 2 | NED | 2 (3) | Recurrent disease (14 mo); died at |
| | | | | | | | | 27 mo |
| 2024 | 60 | M | III: T2N2M0 | 1 | 1 | NED | 2 (3) | Recurrent disease (2 mo); alive |
| 2047 | 16 | M | II: T2N1M0 | 3 | 3 | NED | 2 (3) | Remains in remission (>25 mo); |
| | | | | | | | | alive |
| 894 | 36 | M | III: T3N0M0 | 3 | 2 | Primary site | 2 (2) | CR (44 mo); died at 65 mo |
| 389 | 17 | F | IV: T4N2M0 | 2 | 1 | Primary site | 5 (2) | CR (>53 mo); alive |
| 918 | 16 | M | IV: T4N2M0 | 1 | 1 | Primary site | 2 (2) | PR (12 mo); died at 20 mo |
| 815 | 19 | M | IV: T4N3M0 | 1 | 1 | Primary site | 2 (3) | CRu (>46 mo); alive |
| | | | | | | (unknown | | |
| | | | | | | significance) | | |
| 845 | 11 | M | IV: T4N2M0 | 6 | 2 | Primary site, LN | 1 (1) | PD; died at 12 mo |
| 1042 | 46 | F | II: T2N1M0 | 2 | 2 | Bone | 2 (3) | CR (>68 mo); alive |
| 1046 | 16 | M | IV: T2bN3M0 | 4 | 1 | LN, bone | 2 (3) | SD (2 mo); died at 3 mo |
| 1241 | 18 | M | IV: T4N1M0 | 3 | 2 | Lung (unknown | 2 (3) | CRu (36 mo); alive |
| | | | | | | significance) | | |
| 1902 | 17 | M | III: T3N1M0 | 2 | 1 | Primary site, | 2 (3) | PD; died at 14 mo |
| | | | | | | bone, lung | | |
| 1968 | 34 | M | IV: T4N1M0 | 4 | 2 | Primary site, | 2 (3) | PD; died at 11 mo |
| | | | | | | lung, CNS | | |
| 2019 | 17 | M | IV: T4N3M0 | 3 | 3 | Primary site, | 2 (3) | SD (4 mo); died at 7 mo |
| | | | | | | bone | | |
| 2061 | 32 | M | IV: T0N3M1 | 4 | 1 | Primary, LN, | 2 (3) | PD; died at 16 mo |
| | | | | | | bone, lung | | |
| 2078 | 16 | M | IV: T2N3aM1 | 4 | 2 | LN, bone, soft | 2 (3) | PD; died at 7 mo |
| | | | | | | tissue | | |
| 1668 | 21 | M | IV: T4N2aM0 | 4 | 4 | LN, bone | 3 (3) | PR (7 mo); alive |
| 1976 | 48 | M | I: T1N0M0 | 3 | 1 | Lung | 6 (3) | SD (12 mo); alive |

CNS indieates central nervous system;
CR, complete response;
Cru, complete response undetermined;
CT, chemotherapy regimens;
CTL*, number of CTL infusions;
LN, lymph node;
Mo, months;
NED, no evidence of diseases;
P#, patient identification number;
PD, progressive disease;
PR, partial response;
SD, stable disease;
XRT, radiotherapy regimens.

At the first CTL infusion, the mean patient age was 29.2 years, and most patients had failed 1 or more earlier chemotherapy protocols. Approximately 65% had either active disease or diagnostic scans of unknown significance.

### Example 3

### Characteristics of EBV-CTL Lines

EBV-CTL lines were generated successfully for all 23 study participants. Patients' CTL lines contained a high percentage of CD3-positive T cells [mean, 96.2%; standard deviation (SD), 4.0%]; predominantly CD8 [mean, 83.2%; SD, 13.9%] with a small CD4 component (mean, 7.6%; SD 14.4%). Flow cytometric analysis of memory markers showed mixed populations of CD45RA^{neg} CD27^{pos} CD28^{pos} T cells (mean 34.2%; SD 14.5%) and CD45RAneg CD27^{neg} CD28^{neg} T cells (mean 21.1%; SD 12.3%). Besides T cells, natural killer (NK) cells were present in low numbers (mean, 5.7%; sd, 3.8%). In 4-hour ⁵¹Chromium release cytotoxicity assays, lines from all patients produced significantly greater killing of autologous LCL compared with mismatched LCL, NK cell targets (HSB-2 or K562), or autologous PHA blasts at an effector to target ratio of 20:1 (for all targets Pr0.001). To determine which EBV antigens were recognized by the patients' CTL lines, IFN-γ ELIspot assays were used to measure responses to HLA-restricted peptides derived from EBV antigens. For patients in whom no HLA-restricted peptides were available, peptide mixes were used that contained 15 amino acid peptides scanning the entire length of EBV antigens. T cells specific for immunodominant EBV antigens (lytic or EBNAs 3A, B, C) were present in all cell lines tested (Table 3).

TABLE 3
Specificity of EBV-specific CTL Lines vs. Response

| | Specificity | | | | | Response to Therapy |
|---|---|---|---|---|---|---|
| P# | EBNA1 | EBNA3A-C | LYTIC | LMP1 | LMP2 | (Duration in Months) |
| 729 | Neg | ++ | ++++ | Neg | +++++ | Remains in remission (>82 mths); |
| | | | | | | alive |
| 606 | Neg | ++++ | + | + | ++++ | Recurrent disease (60 mths); |
| | | | | | | alive |
| 697 | ND | ++ | +++ | ND | ++ | Remains in remission (>80 mths); |
| | | | | | | alive |
| 1642 | Neg | + | + | Neg | + | Remains in remission (>42 mths); |
| | | | | | | alive |
| 1946 | +++ | + | + | + | + | Remains in remission (>28 mths); |
| | | | | | | alive |
| 1969 | ++ | ++++ | + | Neg | ++ | Recurrent disease (14 mths); |
| | | | | | | died at 27 mths |
| 2024 | Neg | +++ | ++ | ++ | ++ | Recurrent disease (2 mths); |
| | | | | | | alive |
| 2047 | Neg | ++ | ++++ | Neg | Neg | Remains in remission (>25 mths); |
| | | | | | | alive |
| 894 | ++++ | +++ | +++ | Neg | + | CR (44 mths); died at 65 mths |
| 389 | Neg | +++++ | ++ | Neg | ++ | CR (>53 mths); alive |
| 918 | Neg | Neg | ++ | Neg | Neg | PR (12 mths); died at 20 mths |
| 815 | + | + | ++ | Neg | Neg | CRu (>46 mths); alive |
| 845 | ND | ++ | Neg | Neg | Neg | PD; died at 12 mths |
| 1042 | ND | ND | ++ | Neg | + | CR (>68 mths); alive |
| 1046 | ND | Neg | ++ | + | ++ | SD (2 mths); died at 3 mths |
| 1241 | ++ | +++ | + | Neg | + | CRu (36 mths); alive |
| 1902 | Neg | +++ | ++ | + | + | PD; died at 14 mths |
| 1968 | ND | + | ++ | + | Neg | PD; died at 11 mths |
| 2019 | Neg | ++ | ++++ | + | + | SD (4 mths); died at 7 mths |
| 2061 | Neg | + | ++ | Neg | + | PD; died at 16 mths |
| 2078 | Neg | ++ | ++ | Neg | Neg | PD; died at 7 mths |
| 1668 | Neg | +++ | ++ | Neg | + | PR (7 mths); alive |
| 1976 | Neg | ++ | +++ | Neg | ++ | SD (12 mths); alive |

P#: patient identification number;
*no epitopes; (*) HLA restriction unknown
Frequency:
+++++: >2%;
++++: 1-2%;
+++: 0.5-1%;
++: 0.49-0.1%;
+: <0.1%;
Mths: months;
CR: complete response;
PR: partial response;
CRu: complete response undetermined;
SD: stable disease;
PD: progressive disease;
ND: not done

EBNA 1-specific T cells were present in 5 out of 16 cell lines tested. LMP2-specific T cells were detected in 17 cell lines, but the frequency was less than 0.1% in 9 out of 17. T cells specific for LMP1 were detected at a low frequency in 7 cell lines.

### Example 4

### Safety Of EBV-CTL Infusions

Infusion of autologous EBV-CTL products was not associated with long-term toxicity, although as reported earlier, 1 patient with bulky disease and preexisting facial swelling required a tracheostomy for airway protection after his facial swelling increased 2 days after the first CTL infusion. (Straathof et al., 2005) No inflammatory cells were detected in the needle biopsy of his mass, but CTL administration could not be excluded as a contributing factor to the swelling.

### Example 5

### Immune Responses after EBV-CTL Infusion

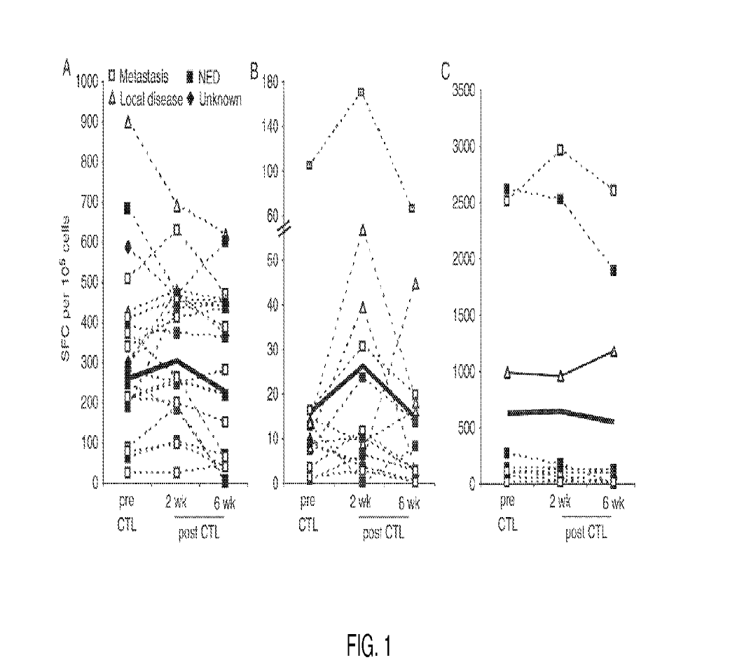

The overall precursor frequency of EBVspecific T cells in patients' peripheral blood was measured using autologous LCL as stimulators in IFN-g ELIspot assays. There was no significant increase 2 and 6 weeks after the first CTL infusion (FIG. 1A). As LCL preferentially activate T cells specific for immunodominant EBV proteins that are absent on NPC tumors, the frequency of T cells specific for LMP2 was determined, a subdominant EBV antigen expressed by NPC. Seventeen patients received CTL lines that contained LMP2-specific T cells and the inventors were able to serially determine the frequency of LMP2-specific CTL in the peripheral blood of 14 using LMP2 peptide ELIspot assays. There was a trend toward increased LMP2-specific CTL at 2 weeks post infusion (P=0.081), which returned to baseline by week 6 (FIG. 1B). As a control, the frequency of CMVpp65-specific T cells were sequentially measured and noted no detectible changes (FIG. 1C). No correlation was observed between disease stage at CTL infusion and the frequency of EBV-specific, LMP2-specific, and CMVspecific T cells. Therefore, infusion of EBV-specific CTL may transiently increase the frequency of LMP2-specific T cells in peripheral blood.

### Example 6

### Clinical Responses and Subgroup Analysis after EBV-CTL Infusion

Clinical responses were measured by comparing imaging studies pre-CTL and post-CTL infusion as detailed above. These data are summarized in Table 4.

TABLE 4
Univariate Cox Regression Analysis

| | PFS | | OS | |
|---|---|---|---|---|
| | Hazard | | Hazard | |
| Variable | Ratio | P | Ratio | P |
| Age at 1st CTL infusion | 0.996 | 0.787 | 0.972 | 0.209 |
| Ethnic Origin | | | | |
| African-American | 0.581 | 0.531 | 0.860 | 0.869 |
| White | 1.492 | 0.515 | 0.858 | 0.841 |
| Other | 1 | | 1 | |
| Stage at Diagnosis | | | | |
| Stage I-III | 1 | | 1 | |
| Stage IV | 1.325 | 0.606 | 2.225 | 0.263 |
| Disease status at first CTL infusion | | | | |
| Remission | | | | |
| Yes | 0.343 | 0.103 | 0.154 | 0.080 |
| No | 1 | | 1 | |
| Local-Regional | | | | |
| Yes | 0.740 | 0.695 | 1.388 | 0.687 |
| No | 1 | | 1 | |
| Unknown | | | | |
| Yes | 0.643 | 0.674 | NA* | |
| No | 1 | | | |
| Metastatic disease | | | | |
| Yes | 3.908 | 0.015 | 5.551 | 0.022 |
| No | 1 | | 1 | |

*No death event occurred.
PFS indicates progression-free survival;
OS, overall survival.

Of the 8 patients treated in remission, 5 remained disease free for 25 to 82 months post-infusion. Of the remaining 3, one had recurrent, biopsy proven, EBV-positive lymph node disease 2 months post CTL infusion. This patient is still alive with indolent disease 34 months post CTL infusion having received no additional therapy. Two patients had recurrent metastatic disease, 14 and 60 months post CTL infusion, respectively. Of 3 patients treated with local recurrent disease, 2 have had CRs for more than 44 and more than 53 months. The third patient had a partial response that persisted for 12 months. A fourth patient had imaging findings of unknown significance before CTL infusion, and these resolved post infusion. He remains without evidence of disease (CRu) at more than 46 months.

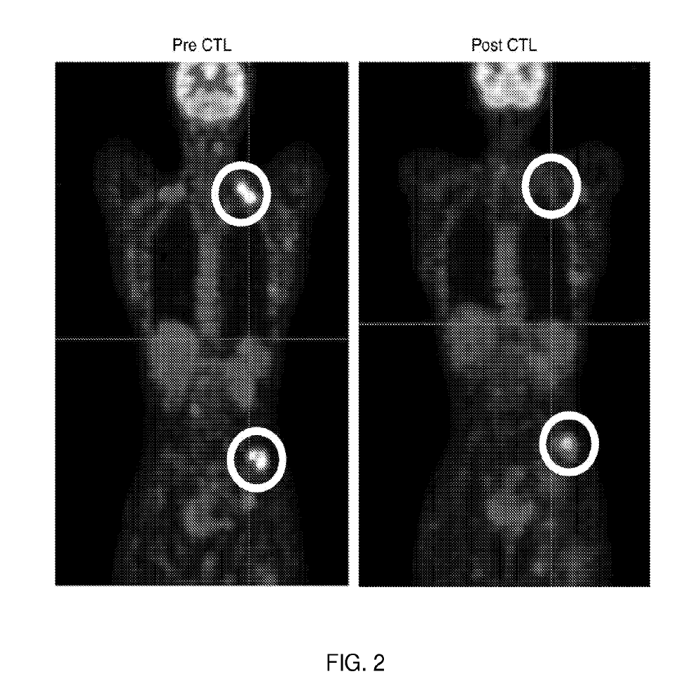

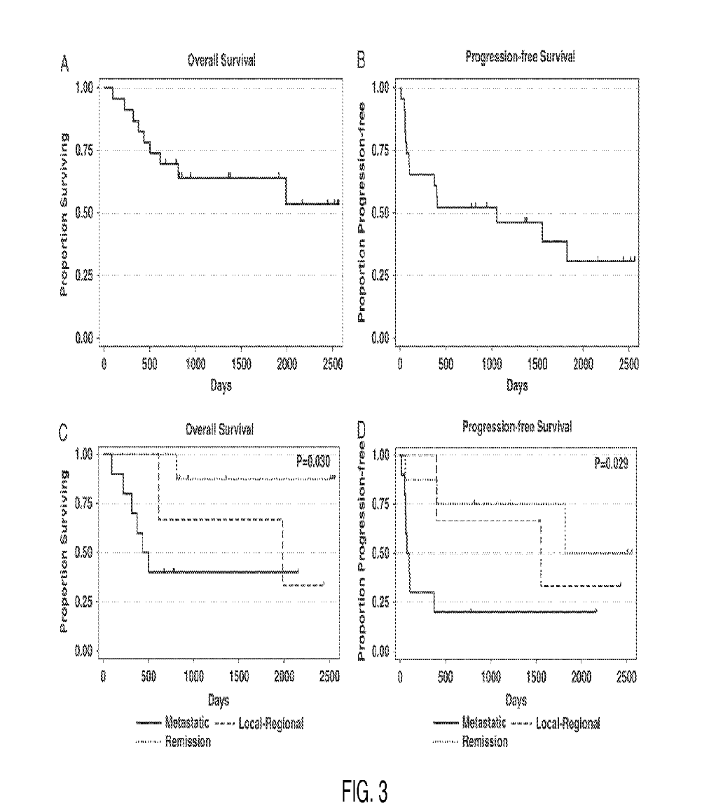

In the 11 patients with metastatic disease, 1 patient (1042) has had a durable response for more than 68 months. This patient was initially reported at having stable disease more than 14 months (Straathof et al., 2005); starting 26 months post CTL she had no evidence of disease and remains disease free for more than 68 months post CTL infusion. In this analysis she is therefore reported as having a CR. Patient 1241 had a CRu for 36 months. The latter then had a local, EBV-negative recurrence. One patient had a significant PR with resolution of PET positive lymph nodes and a significant decrease in a metastatic bone lesion (FIG. 2). Two other patients had SD for 2 and 12 months, respectively. Six patients had progressive disease. For the entire study cohort, the median time to progression was 1059 days with progression-free survival (PFS) rates of 65% at 1-year and 52% at 2 years (FIG. 3).

The overall survival (OS) was 87% at 1 year and 70% at 2 years. Metastatic disease was associated with an increased risk of disease progression (HR: 3.91, P=0.015) and decreased overall survival (HR: 5.55, P=0.022). No significant association was observed for patients with locoregional disease. PFS and OS did not correlate with ethnicity, stage at diagnosis (Table 4), or CTL-specificity (Table 5).

TABLE 5

| | EBNA1 | | | | EBNA 3A-C | | | | LYTIC | | |
|---|---|---|---|---|---|---|---|---|---|---|---|
| | Not | | | | Not | | | | Not | | |
| | specific | Specific | ND^{†} | | specific | Specific | ND^{†} | | specific | Specific | |
| | n(%) | n(%) | n(%) | P^{ζ} | n(%) | n(%) | n(%) | P^{ζ} | n(%) | n(%) | P^{ζ} |
| Response | | | | | | | | | | | |
| CR | 1(12.5) | 3(100) | 1(25) | 0.35 | 0(0) | 4(33.3) | 1(100) | 0.36 | 0(0) | 5(35.7) | 1.00 |
| PR | 2(28) | 0(0) | 0(0) | | 1(50) | 1(8.3) | 0(0) | | 0(0) | 2(14.3) | |
| SD | 2(25) | 0(0) | 1(25) | | 1(50) | 2(16.7) | 0(0) | | 0(0) | 3(21.4) | |
| PD | 3(37.5) | 0(0) | 2(50) | | 0(0) | 5(41.7) | 0(0) | | 1(100) | 4(28.5) | |
| Response* | | | | | | | | | | | |
| CR + PR | 6(62.5) | 0(0) | 3(75) | 0.10 | 1(50) | 7(68.3) | 0(0) | 0.71 | 1(100) | 7(60) | 1.00 |
| SD + PD | 3(37.5) | 3(100) | 1(25) | | 1(50) | 5(41.7) | 1(100) | | 0(0) | 7(50) | |

| | LMP1 | | | LMP2 | | |
|---|---|---|---|---|---|---|
| | Not | | | Not | | |
| | specific | Specific | | specific | Specific | |
| | n(%) | n(%) | P^{ζ} | n(%) | n(%) | P^{ζ} |
| Response | | | | | | |
| CR | 5(45.5) | 0(0) | 0.23 | 1(20) | 4(40) | 0.38 |
| PR | 2(18.2) | 0(0) | | 1(20) | 1(10) | |
| SD | 1(9.1) | 2(80) | | 0(0) | 3(30) | |
| PD | 3(27.3) | 2(50) | | 3(60) | 2(20) | |
| Response* | | | | | | |
| CR + PR | 4(36.4) | 4(100) | 0.08 | 3(60) | 5(50) | 1.00 |
| SD + PD | 7(63.6) | 0(0) | | 2(40) | 6(50) | |

*N: SD or PD, Y: CH, PH
^{†}Not determined
^{ζ}Fisher's exact test.

### Example 7

### Significance of Certain Embodiments of the Invention

In studies provided herein, the safety and efficacy of EBV-CTL in patients with EBV-positive NPC was evaluated. Twenty-three NPC patients were treated with EBV-specific CTLs and followed long-term. EBV-CTLs had potent antitumor activity in patients with locoregional disease, whereas similar effects were limited in patients with metastatic disease. Nonetheless, PFS was 65% at 1 year and 52% at 2 years, whereas overall survival was 87% at 1 year and 70% at 2 years. In this limited cohort of patients, clinical outcome did not correlate with specificity or expansion of adoptively transferred CTL lines. Although patients with nonbulky, recurrent, locoregional NPC have a long-term survival rate greater than 60% with chemoradiation, (Chua et al., 2005a; Chua et al., 2005b) these therapies are often mutilating and associated with significant acute and longterm complications profoundly diminishing quality-of-life.

The results indicate that EBV-CTLs may have comparable efficacy and greatly reduced toxicities. More extensive studies will be required to validate the therapeutic advantages of autologous EBV-CTLs in this subset of patients. The outcome for patients with recurrent, metastatic disease remains poor. Currently, there is no accepted standard second-line therapy for this group of patients. Since 2002, there have been at least 7 Phase II clinical studies in which patients with relapsed and/or metastatic NPC were treated with systemic chemotherapy. (Ngan et al., 2002; McCarthy et al., 2002; Chua et al., 2003; Zhang et al., 2008; Poon et al., 2005; Chan et al., 2005; Chua et al., 2008) Four of the regimens used single agents (irinotecan, capecitabine, gemcitabine, or gefitinib) whereas the remainder gave 2-drug, platinum-based therapy. The overall CR and PR rates in these studies were only 4.4% (0% to 20.5%) and 27% (14.3% to 78%), respectively. The median time to progression was variable (81 to 318 d), most likely owing to selection bias of study participants. Unfortunately, the EBV-CTLs had an equally limited antitumor activity in patients with metastatic disease (CR rate 10%; PR rate 10%), with a median PFS of 74 days. One of the patients with recurrent disease had an EBV-negative tumor.

Antigen-negative recurrences have been reported after the infusion of T-cell clones. (Yee et al., 2002) We have also seen EBV-negative recurrences in 2 patients treated in ongoing protocols with polyclonal EBV-specific CTLs for EBV-positive lymphomas. In addition, earlier a patient with EBV-positive lymphoproliferative disease, who had failed EBV-specific T-cell therapy, in which the tumor virus had deleted immunodominant EBV epitopes. (Gottschalk et al., 2001) Thus immune escape can occur even in instances when polyclonal antigen-specific T cells are infused.

NPC is classified as an EBV Type II latency-associated tumor as it expresses only a limited range of EBVassociated antigens. (Raab-Traub, 2002) In some cases, the greatest antitumor activity would could be associated with those lines expressing the highest frequency of effector cells specific for the 3 EBV antigens expressed by these tumors: LMP1, LMP2, or EBNA 1. Although this cohort of patients was small, there was no positive correlation between CTL specificity and antitumor activity. Activity was even seen after the infusion of lines with an absence of reactivity with LMP1 and EBNA1 While the majority of T-cell lines contained CTLs specific for LMP2, their overall frequency was low and there was no correlation between the levels seen and the antitumor activity observed. Nonetheless it seems likely that one requirement for increasing the effectiveness of autologous EBV-CTLs in metastatic NPC will be to augment the frequency of EBNA1-specific, LMP1-specific, and/or LMP2-specific T cells in the CTL product. (Gottschalk et al., 2003; Fogg et al., 2009; Bollard et al., 2004; Lutzky et al., 2009) Such benefits are certainly observed when LMP 1/LMP2 specific CTLs are used to treat EBV-positive lymphomas, which are also predominantly Type II latency tumors. (Bollard et al., 2007) An alternative strategy for augmenting activity in metastatic disease is to induce tumor expression of more immunodominant EBV antigens by using chemotherapeutic agents, histone deacetylase, or proteasome inhibitors. (Feng et al., 2002; Feng et al., 2006) Preclinical data have shown benefit from this type of approach, but clinical experience is thus far limited. (Chan et al., 2004; Perrine et al., 2007)

There was limited in vivo expansion of EBV-CTLs in peripheral blood of patients after infusion. IFN-γ ELIspot assays showed only transient 2-fold increases in LMP2-specific T cells. Lymphodepletion before T-cell infusion seems to enhance the expansion and persistence of infused cells, and may also be a valuable approach for NPC patients. (Louis et al., 2009; Dudley et al., 2008) The NPC patients enrolled on this study were not lymphopenic (mean absolute lymphocyte count: 829/mL; SD 373/mL), and thus lympodepletion would be an option to improve in vivo, postinfusion expansion. In several patients there was a slight, nonsignificant decrease in the precursor frequency of EBV-CTL. This most likely reflects the physiological fluctuations, which has been observed in earlier studies. (Louis et al., 2009; Bollard et al., 2007).

In summary, treatment of patients with relapsed or refractory EBV-positive NPC with EBV-specific CTL is safe and is associated with significant, long-term clinical benefit, at least in patients with locoregional disease.

### REFERENCES

All patents and publications mentioned in this specification are indicative of the level of those skilled in the art to which the invention pertains. All patents and publications herein are incorporated by reference to the same extent as if each individual publication was specifically and individually indicated to be incorporated by reference in their entirety.
Ayan I, Kaytan E, Ayan N. Childhood nasopharyngeal carcinoma: from biology to treatment. Lancet Oncol. 2003; 4:13-21.
Bollard C M, Aguilar L, Straathof K C, et al. Cytotoxic T lymphocyte therapy for Epstein-Barr Virus+Hodgkin's disease. J Exp Med. 2004; 200:1623-1633.
Bollard C M, Gottschalk S, Leen A M, et al. Complete responses of relapsed lymphoma following genetic modification of tumor-antigen presenting cells and T-lymphocyte transfer. Blood. 2007; 110:2838-2845.
Bollard C M, Straathof K C, Huls M H, et al. The generation and characterization of LMP2-specific CTLs for use as adoptive transfer from patients with relapsed EBV-positive Hodgkin disease. J. Immunother. 2004; 27:317-327.
Chan A T, Hsu M M, Goh B C, et al. Multicenter, phase II study of cetuximab in combination with carboplatin in patients with recurrent or metastatic nasopharyngeal carcinoma. J Clin Oncol. 2005; 23:3568-3576.
Chan A T, Tao Q, Robertson K D, et al. Azacitidine induces demethylation of the Epstein-Barr virus genome in tumors. J Clin Oncol. 2004; 22:1373-1381.
Chan A T, Teo P M, Johnson P J. Nasopharyngeal carcinoma. Ann Oncol. 2002; 13:1007-1015.
Chua D T, Sham J S, Au G K. A phase II study of capecitabine in patients with recurrent and metastatic nasopharyngeal carcinoma pretreated with platinum-based chemotherapy. Oral Oncol. 2003; 39:361-366.
Chua D T, Sham J S, Au G K. Induction chemotherapy with cisplatin and gemcitabine followed by reirradiation for locally recurrent nasopharyngeal carcinoma. Am J Clin Oncol. 2005; 28:464-471.
Chua D T, Sham J S, Leung L H, et al. Re-irradiation of nasopharyngeal carcinoma with intensity-modulated radiotherapy. Radiother Oncol. 2005; 77:290-294.
Chua D T, Wei W I, Wong M P, et al. Phase II study of gefitinib for the treatment of recurrent and metastatic nasopharyngeal carcinoma. Head Neck. 2008; 30:863-867.
Comoli P, Pedrazzoli P, Maccario R, et al. Cell therapy of stage 1V nasopharyngeal carcinoma with autologous Epstein-Barr Virus-targeted cytotoxic T lymphocytes. J Clin Oncol. 2005; 23:8942-8949.
Cooper J S. Current and future therapy of nasopharyngeal cancer. Int J Radiat Oncol Biol Phys. 1997; 37:973-974.
Dudley M E, Yang J C, Sherry R, et al. Adoptive cell therapy for patients with metastatic melanoma: evaluation of intensive myeloablative chemoradiation preparative regimens. J Clin Oncol. 2008; 26:5233-5239.
Feng W H, Israel B, Raab-Traub N, et al. Chemotherapy induces lytic EBV replication and confers ganciclovir susceptibility to EBV-positive epithelial cell tumors. Cancer Res. 2002; 62:1920-1926.
Feng W H, Kenney S C. Valproic acid enhances the efficacy of chemotherapy in EBV-positive tumors by increasing lytic viral gene expression. Cancer Res. 2006; 66:8762-8769.
Fogg M H, Wirth L J, Posner M, et al. Decreased EBNA-1-specific CD8+ T cells in patients with Epstein-Ban Virusassociated nasopharyngeal carcinoma. Proc Natl Acad Sci USA. 2009; 106:3318-3323.
Gottschal k S, Edwards O L, Sili U, et al. Generating CTL against the subdominant Epstein-Barr Virus LMP1 antigen for the adoptive Immunotherapy of EBV-associated malignancies. Blood. 2003; 101:1905-1912.
Gottschalk S, Ng C Y C, Smith C A, et al. An Epstein-Barr Virus deletion mutant that causes fatal lymphoproliferative disease unresponsive to virus-specific T-cell therapy. Blood. 2001; 97:835-843.
Hislop A D, Taylor G S, Sauce D, et al. Cellular responses to viral infection in humans: lessons from Epstein-Ban Virus. Annu Rev Immunol. 2007; 25:587-617.
Khanna R, Burrows S R. Role of cytotoxic T lymphocytes in Epstein-Barr Virus-associated diseases. Annu Rev Microbiol. 2000; 54:19-48.
Lee C C, Huang T T, Lee M S, et al. Clinical application of tumor volume in advanced nasopharyngeal carcinoma to predict outcome. Radiat Oncol. 2010; 5:20.
Louis C U, Paulino A C, Gottschalk S, et al. A single institution experience with pediatric nasopharyngeal carcinoma: high incidence of toxicity associated with platinum-based chemotherapy plus IMRT. J Pediatr Hematol Oncol. 2007; 29:500-505.
Louis C U, Straathof K, Bollard C M, et al. Enhancing the in vivo expansion of adoptively transferred EBV-specific CTL with lymphodepleting CD45 monoclonal antibodies in NPC patients. Blood. 2009; 113:2442-2450.
Lutzky V P, Corban M, Heslop L, et al. A novel approach to the formulation of an EBV antigen-based nasopharyngeal carcinoma vaccine. J. Virol. 2009.
McCarthy J S, Tannock I F, Degendorfer P, et al. A Phase II trial of docetaxel and cisplatin in patients with recurrent or metastatic nasopharyngeal carcinoma. Oral Oncol. 2002; 38: 686-690.
Mould R F, Tai T H. Nasopharyngeal carcinoma: treatments and outcomes in the 20th century. Br J. Radiol. 2002; 75: 307-339.
Ngan R K, Yiu H H, Lau W H, et al. Combination gemcitabine and cisplatin chemotherapy for metastatic or recurrent nasopharyngeal carcinoma: report of a phase II study. Ann Oncol. 2002; 13:1252-1258.
Perrine S P, Hermine 0, Small T, et al. A phase 1/2 trial of arginine butyrate and ganciclovir in patients with Epstein-Barr virus-associated lymphoid malignancies. Blood. 2007; 109: 2571-2578.
Poon D, Chowbay B, Cheung Y B, et al. Phase II study of irinotecan (CPT-11) as salvage therapy for advanced nasopharyngeal carcinoma. Cancer. 2005; 103:576-581.
Raab-Traub N. Epstein-Barr Virus in the pathogenesis of NPC. Semin Cancer Biol. 2002; 12:431-441.
Rooney C M, Smith C A, Ng C Y C, et al. Infusion of cytotoxic T cells for the prevention and treatment of Epstein-Barr Virusinduced lymphoma in allogeneic transplant recipients. Blood. 1998; 92:1549-1555.
Rosenberg S A, Dudley M E. Adoptive cell therapy for the treatment of patients with metastatic melanoma. Curr Opin Immunol. 2009; 21:233-240.
Rosenberg S A, Restifo N P, Yang J C, et al. Adoptive cell transfer: a clinical path to effective cancer immunotherapy. Nat Rev Cancer. 2008; 8:299-308.
Straathof K C, Bollard C M, Popat U, et al. Treatment of nasopharyngeal carcinoma with Epstein-Barr Virus-specific T lymphocytes. Blood. 2005; 105:1898-1904.
Straathof K C, Leen A M, Buza E L, et al. Characterization of latent membrane protein 2 specificity in CTL lines from patients with EBV-positive nasopharyngeal carcinoma and lymphoma. J. Immunol. 2005; 175:4137-4147.
Therasse P, Arbuck S G, Eisenhauer E A, et al. New guidelines to evaluate the response to treatment in solid tumors. European Organization for Research and Treatment of Cancer, National Cancer Institute of the United States, National Cancer Institute of Canada. J Natl Cancer Inst. 2000; 92:205-216.
Wang C C, Chen M L, Hsu K H, et al. Second malignant tumors in patients with nasopharyngeal carcinoma and their association with Epstein-Barr Virus. Int J. Cancer. 2000; 87: 228-231.
Yee C, Thompson J A, Byrd D, et al. Adoptive T-cell therapy using antigen-specific CD8+ T-cell clones for the treatment of patients with metastatic melanoma: in vivo persistence, migration, and antitumor effect of transferred T cells. Proc Natl Acad Sci USA. 2002; 99:16168-16173.
Zhang L, Zhang Y, Huang P Y, et al. Phase II clinical study of gemcitabine in the treatment of patients with advanced nasopharyngeal carcinoma after the failure of platinumbased chemotherapy. Cancer Chemother Pharmacol. 2008; 61: 33-38.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A method of treating an individual with locoregional nasopharyngeal carcinoma, comprising the step of administering to the individual an effective amount of cytotoxic T lymphocytes (CTLs) that target one or more Epstein Barr Virus (EBV) antigens.

2. The method of claim 1, wherein there are multiple administrations of the CTLs.

3. The method of claim 1, wherein the EBV antigens are selected from the group consisting of LMP2, LMP1, EBNA, and a combination thereof.

4. The method of claim 1, wherein the individual is being treated or has been treated with an additional cancer therapy.
